(19) United States
(12) Patent Application Publication
(10) Pub. No.: US 2010/0273834 A1
(43) Pub. Date: Oct. 28, 2010

(54) TREATMENT OF ONYCHOMYCOSIS AND RELATED COMPOSITIONS INCLUDING UREA

(76) Inventor: Reza Babapour, Beverly Hills, CA (US)

Correspondence Address: KAREN S. CANADY;CANADY & LORTZ LLP, 4201 Wilshire BLV Suite 622, LOS ANGELES, CA, 90010, US

(21) Appl. No.: 12/766,790

(22) Filed: Apr. 23, 2010

Related U.S. Application Data
(60) Provisional application No. 61/214,500, filed on Apr. 25, 2009.

(51) Int. Cl. A61K 31/4418; A61P 31/10

(52) U.S. Cl. 514/345

(57) ABSTRACT

The present invention generally relates to the treatment of onychomycosis. More specifically, it relates to combination therapies for the treatment of onychomycosis and related compositions. In a composition aspect, the present invention provides a composition for the treatment of onychomycosis, wherein the composition includes Ciclopirox and urea. In a method aspect, the present invention provides a method for treating onychomycosis in a patient suffering from the disease. The method involves topically administering a composition to at least one toe or fingernail of the patient and the composition includes Ciclopirox and urea.

This application claims the benefit of U.S. provisional patent application No. 61/214,500, filed Apr. 25, 2009, the entire contents of which are incorporated herein by reference.

### Field of the Invention

The present invention generally relates to the treatment of onychomycosis. More specifically, it relates to combination therapies for the treatment of onychomycosis and related compositions that include urea.

### BACKGROUND OF THE INVENTION

Ciclopirox (PENLAC™, Dermik) 8% solution has been approved for the treatment of onychomycosis in the United States and Canada; it is the only topical antifungal approved for such treatment in Canada. While Ciclopirox shows a degree of efficacy against onychomycosis for certain patient populations, it has proven not to be universally effective against the illness.

Accordingly, there is still a need for the development of new onychomycosis treatments and compositions that are related to those treatments.

### SUMMARY OF THE INVENTION

The present invention generally relates to the treatment of onychomycosis. More specifically, it relates to combination therapies for the treatment of onychomycosis and related compositions.

In a composition aspect, the present invention provides a composition for the treatment of onychomycosis, wherein the composition includes Ciclopirox and urea.

In a method aspect, the present invention provides a method for treating onychomycosis in a patient suffering from the disease. The method involves topically administering a composition to at least one toe or fingernail of the patient, and the composition includes Ciclopirox and urea.

### DETAILED DESCRIPTION OF THE INVENTION

Definitions

“Ciclopirox” refers to 6-cyclohexyl-1-hydroxy-4-methyl-2(1H)-pyridone.

“Ciclopirox olamine” refers to the 2-aminoethanol salt of Ciclopirox.

The present invention relates to combination therapies for the treatment of onychomycosis and related compositions. The compositions include Ciclopirox and urea.

The combination therapies involve the topical application of compositions according to the present invention and optionally include the oral administration of an antifungal agent.

The composition may further optionally include a penetration enhancer such as acetyl cysteine. (For a discussion of penetration enhancers and penetration synergists, see U.S. Pat. No. 6,042,845, which is incorporated-by-reference into this document for all purposes.)

The composition is typically a solution, lotion, cream, emollient, foam, ointment, lacquers, shampoo or spray which is 8% in Ciclopirox, although any concentration that produces a desirable pharmaceutical effect is suitable. Nonlimiting examples of other Ciclopirox concentrations include a 5%, 6%, 7%, 9%, 10%, and 11% solution or cream.

The composition typically includes at least 10% urea. Nonlimiting examples of other urea concentrations include at least 20% urea, at least 25% urea, at least 30% urea, at least 35% urea, at least 40% urea, at least 45% urea, at least 50% urea, at least 55% urea and at least 60% urea.

A nonlimiting list of other agents that may be included in the composition is as follows: cetyl alcohol; cocamide DEA; lactic acid; mineral oil; myristyl alcohol; octyldodecanol; polysorbate 60; purified water; sorbitan monostearate; stearyl alcohol; and benzyl alcohol (1%) as a preservative.

The treatment of onychomycosis involves the topical application of a composition of the present invention to a toe or fingernail of a human or other animal presenting symptoms of the disease.

The treatment may further include the oral administration of an antifungal in conjunction with the topical administration of the composition to a target toe or fingernail. A nonlimiting example of such a regimen is the topical application of a composition including Ciclopirox and urea in conjunction with the oral administration of itraconazole. Nonlimiting examples of antifungals include: terbinafine; itraconazole; ketoconazole; fluconazole; derivatives of fluconazole; oxiconazole; sulconazole; clotrimazole;

miconazole; econazole; azanidazole; bifonazole; butoconazole; chlormidazole; fenticonazole; imazalil; isoconazole; neticonazole; sertaconazole; tioconazole; naftifine; griseofulvin; amorolfine; and sodium pyrithione.

Where the oral administration of an antifungal is used, the antifungal is provided at a significantly lower dose than typically administered for the treatment of onychomycosis—e.g., 90%, 80%, 70%, 60%, 50%, 40%, 30%, 20% or 10% of the typically administered dose.

One objective for the treatment involving the oral administration of a reduced amount of an antifungal agent is the significant reduction of patient side effects, which typically accompany the oral administration of certain antifungal agents—e.g., nausea, abdominal pain, rash, cardiotoxicity and hepatotoxicity.

For instance, using standard metrics for determining the severity of nausea, abdominal pain, rash, cardiotoxicity or hepatotoxicity, one will typically see at least a 10% reduction in side effect severity utilizing the combination therapy employing conjunctive oral administration of an antifungal agent according to the present invention vis-à-vis the simple oral administration of the antifungal agent at a therapeutically effective dose. Oftentimes, one will see at least a 20% reduction, 30% reduction, 40% reduction or 50% reduction in side effect severity.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A composition for the treatment of onychomycosis, wherein the composition comprises: Ciclopirox and urea.

2. The composition according to claim 1, wherein the concentration of Ciclopirox in the composition is 5%, 6%, 7%, 8%, 9%, 10% or 11%.

3. The composition according to claim 2, wherein the composition is a solution, cream, emollient, foam, ointment, lacquer or spray.

4. The composition according to claim 3, wherein the composition contains at least 10% urea.

5. The composition according to claim 4, wherein the composition contains at least 20% urea, at least 25% urea, at least 30% urea, at least 35% urea, at least 40% urea, at least 45% urea, at least 50% urea, at least 55% urea or at least 60% urea.

6. The composition according to claim 5, wherein the composition further includes at least one agent selected from a list of agents consisting of: cetyl alcohol; cocamide DEA; lactic acid; mineral oil; myristyl alcohol; acetyl cysteine; octyldodecanol; polysorbate 60; purified water; sorbitan monostearate; stearyl alcohol; and benzyl alcohol (1%).

7. A method for treating onychomycosis in a patient suffering from the disease, wherein the method comprises topically administering a composition to at least one toe or fingernail of the patient, wherein the composition comprises Ciclopirox and urea.

8. The method according to claim 7, wherein the concentration of Ciclopirox in the composition is 5%, 6%, 7%, 8%, 9%, 10% or 11%.

9. The method according to claim 8, wherein the composition is a solution, cream, emollient, foam, ointment, lotion, shampoo, lacquer or spray.

10. The method according to claim 9, wherein the composition contains at least 10% urea.

11. The method according to claim 10, wherein the composition contains at least 20% urea, at least 25% urea, at least 30% urea, at least 35% urea, at least 40% urea, at least 45% urea, at least 50% urea, at least 55% urea, or at least 60% urea.
